(19) United States
(12) Patent Application Publication
(10) Pub. No.: US 2019/0216431 A1
(43) Pub. Date: Jul. 18, 2019

(54) ULTRASOUND IMAGING APPARATUS AND METHOD OF CONTROLLING THE SAME

(71) Applicant: SAMSUNG MEDISON CO., LTD., Hongcheon-gun (KR)

(72) Inventor: Dong-hee LEE, Seongnam-si (KR)

(73) Assignee: SAMSUNG MEDISON CO., LTD., Hongcheon-gun (KR)

(21) Appl. No.: 16/144,553

(22) Filed: Sep. 27, 2018

(30) Foreign Application Priority Data
Jan. 16, 2018 (KR) 10-2018-0005509

(51) Int. Cl. A61B 8/00

(52) U.S. Cl. CPC A61B 8/467; A61B 8/54; A61B 8/465; A61B 8/464; A61B 8/4254

(57) ABSTRACT

Provided are an ultrasound imaging apparatus and a method of controlling the same. The ultrasound imaging apparatus may: receive an input for selecting a patient in a stored patient list; determine an auxiliary information output form according to a disability type of the selected patient; executing a diagnosis process corresponding to a diagnosis item of the selected patient; and outputting in real time, in the determined auxiliary information output form, auxiliary information corresponding to a progression stage of the executed diagnosis process, wherein when the selected patient is a person with auditory disability, a sign language is determined as the auxiliary information output form.

### CROSS-REFERENCE TO RELATED APPLICATION

This application is based on and claims priority under 35 U.S.C. § 119 to Korean Patent Application No. 10-2018-0005509, filed on Jan. 16, 2018, in the Korean Intellectual Property Office, the disclosure of which is incorporated by reference herein in its entirety.

### BACKGROUND

### 1. Field

The disclosure relates to an ultrasound imaging apparatus and a method of controlling the same.

### 2. Description of Related Art

Ultrasound imaging apparatuses transmit ultrasound signals generated by transducers of a probe to an object and receive information about signals reflected from the object, thereby obtaining at least one image of an internal part (e.g., soft tissue or blood flow) of the object.

Ultrasound imaging apparatuses may obtain ultrasound images respectively corresponding to steps of an ultrasound diagnosis process and may provide the obtained ultrasound images to a user.

### SUMMARY

Provided are a system and a method of automatically guiding an ultrasound diagnosis process to a patient.

Provided are a system and a method of automatically guiding an ultrasound diagnosis process to disabled people who are difficult for an examiner to guide through the ultrasound diagnosis process.

Additional aspects will be set forth in part in the description which follows and, in part, will be apparent from the description, or may be learned by practice of the presented embodiments.

In accordance with an aspect of the disclosure, an ultrasound imaging apparatus includes: a storage configured to store a patient list and store a sign language, a subtitle, and a voice as auxiliary information corresponding to a diagnosis process; an input interface configured to receive an input for selecting a patient in the patient list; at least one processor configured to determine an auxiliary information output form according to a disability type of the selected patient, and execute a diagnosis process corresponding to a diagnosis item of the selected patient; and an output interface configured to output in real time, in the determined auxiliary information output form, auxiliary information corresponding to a progression stage of the executed diagnosis process, wherein, when the selected patient is a person with auditory disability, the sign language is determined as the auxiliary information output form.

The input interface may be further configured to receive an input regarding patient information, and the storage is further configured to store the patient information in the patient list based on the input regarding the patient information, wherein the patient information includes at least one or a combination of information about a disability type of the patient, information about a language used by the patient, and information about a caregiver of the patient.

The input interface may be further configured to receive an input for changing an output form of the auxiliary information, and the at least one processor may be further configured to change the output form of the auxiliary information based on the input for changing the output form of the auxiliary information, wherein the input for changing the output form of the auxiliary information includes an input for stopping outputting of the auxiliary information.

When the determined auxiliary information output form is the sign language, the subtitle, or a combination thereof, the input interface may be further configured to receive an input regarding at least one or a combination of an output position, an output size, and an output transparency of the auxiliary information corresponding to the progression stage of the executed diagnosis process, and the output interface is further configured to output the auxiliary information corresponding to the progression stage of the executed diagnosis process based on the input regarding the at least one or the combination of the output position, the output size, and the output transparency of the auxiliary information.

When the selected patient is a non-disabled person, the input interface may be further configured to receive a user input regarding the auxiliary information output form, the at least one processor may be further configured to change the determined auxiliary information output form based on the user input regarding the auxiliary information output form, and the output interface may be further configured to output in real time, in the changed auxiliary information output form, the auxiliary information corresponding to the progression stage of the executed diagnosis process.

The ultrasound imaging apparatus may further include an auxiliary output interface configured to output in real time, in the determined auxiliary information output form, the auxiliary information corresponding to the progression stage of the executed diagnosis process.

The ultrasound imaging apparatus may further include a communicator configured to transmit in real time, in the determined auxiliary information output form, the auxiliary information corresponding to the progression stage of the executed diagnosis process to a mobile terminal of the selected patient or a mobile terminal of a caregiver of the selected patient.

The output interface may be further configured to, whenever an event occurs in the progression stage of the executed diagnosis process, output in real time, in the determined auxiliary information output form, auxiliary information corresponding to the event, wherein the event includes at least one or a combination of a freeze, a measurement, a caliper, and a report.

The input interface may be further configured to receive a user input regarding the auxiliary information corresponding to the diagnosis process, and the storage may be further configured to store the sign language, the subtitle, and the voice as the auxiliary information corresponding to the diagnosis process based on the user input regarding the auxiliary information corresponding to the diagnosis process, wherein the user input regarding the auxiliary information corresponding to the diagnosis process includes at least one or a combination of an input for modifying the auxiliary information stored in the storage and an input for adding the auxiliary information corresponding to the diagnosis process.

The input interface may be further configured to receive an input including at least one or a combination of a voice and characters regarding a diagnosis situation and a diagnosis result, the diagnosis situation and the diagnosis result each corresponding to the progression stage of the diagnosis process, the at least one processor may be further configured to generate the sign language, the subtitle, or the voice as diagnosis information, based on the input, and the output interface may be further configured to output the diagnosis information in the determined auxiliary information output form in real time.

The ultrasound imaging apparatus may further include a communicator configured to transmit the generated diagnosis information in the determined auxiliary information output form to a mobile terminal of the selected patient or a mobile terminal of a caregiver of the selected patient.

In accordance with another aspect of the disclosure, a method of controlling an ultrasound imaging apparatus includes: receiving an input for selecting a patient in a stored patient list; determining an auxiliary information output form according to a disability type of the selected patient; executing a diagnosis process corresponding to a diagnosis item of the selected patient; and outputting in real time, in the determined auxiliary information output form, auxiliary information corresponding to a progression stage of the executed diagnosis process, wherein, when the selected patient is a person with auditory disability, a sign language is determined as the auxiliary information output form.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram illustrating a configuration of an ultrasound diagnosis apparatus according to an embodiment;

FIGS. 2A, 2B, and 2C are views illustrating ultrasound diagnosis apparatuses according to an embodiment;

FIG. 3 is a block diagram illustrating a structure of an ultrasound imaging apparatus according to an embodiment;

FIG. 4 is a flowchart of a method of automatically guiding ultrasound diagnosis performed by the ultrasound imaging apparatus, according to an embodiment;

FIG. 5 is a diagram illustrating a process by which the ultrasound imaging apparatus receives a patient's selection, according to an embodiment;

FIG. 6 is a diagram illustrating an example where, when a selected patient is a person with auditory disability, the ultrasound imaging apparatus automatically outputs auxiliary information, according to an embodiment;

FIGS. 7A and 7B are diagrams illustrating an example where the ultrasound imaging apparatus changes at least one of a position, a size, and a transparency of output auxiliary information, according to an embodiment;

FIGS. 8A and 8B are diagrams illustrating an example where, when a selected patient is a non-disabled person, the ultrasound imaging apparatus outputs auxiliary information corresponding to an ultrasound diagnosis process, according to an embodiment;

FIG. 9 is a diagram illustrating an example where the ultrasound imaging apparatus outputs auxiliary information corresponding to an ultrasound diagnosis process, according to an embodiment;

FIG. 10 is a diagram illustrating an example where the ultrasound imaging apparatus outputs auxiliary information corresponding to an event occurring in an ultrasound diagnosis process, according to an embodiment; and

FIG. 11 is a flowchart of a method by which the ultrasound imaging apparatus changes an auxiliary information output form, according to an embodiment.

### DETAILED DESCRIPTION

The principle of the present disclosure is described and embodiments are disclosed so that the scope of the present disclosure is clarified and clarified and one of ordinary skill in the art to which the present disclosure pertains implements the present disclosure. The disclosed embodiments may have various forms.

Throughout the specification, the same reference numerals denote the same elements. In the present specification, all elements of embodiments are not described, but general matters in the technical field of the present disclosure or redundant matters between embodiments will not be described. The term ‘module’ or ‘unit’ used herein may be implemented using one or more combinations of hardware, software, and firmware. According to embodiments, a plurality of ‘modules’ or ‘units’ may be implemented using a single element, or a single ‘module’ or ‘unit’ may include a plurality of elements.

The operational principle and embodiments of the present disclosure will now be described with reference to the accompanying drawings.

Throughout the specification, an image may include a medical image obtained by a medical imaging apparatus such as a magnetic resonance imaging (MRI) apparatus, a computed tomography (CT) apparatus, an ultrasound imaging apparatus, or an X-ray apparatus.

Through the present specification, the term ‘object’ is a thing to be imaged, and may include a human, an animal, or a part of a human or an animal. For example, the object may include a part of a body (e.g., an organ), a phantom, or the like.

Throughout the specification, the term “ultrasound image” refers to an image of an object processed based on ultrasound signals transmitted to the object and reflected therefrom.

Embodiments will now be described in detail with reference to the drawings.

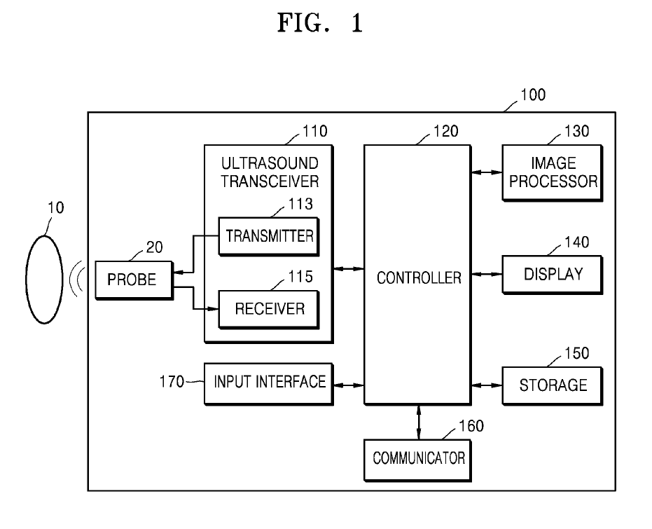

FIG. 1 is a block diagram illustrating a configuration of an ultrasound diagnosis apparatus 100 according to an embodiment.

Referring to FIG. 1, the ultrasound diagnosis apparatus 100 may include a probe 20, an ultrasound transceiver 110, a controller 120, an image processor 130, a display 140, a storage 150, a communicator 160, and an input interface 170.

The ultrasound diagnosis apparatus 100 may be a cart-type or a portable-type ultrasound diagnosis apparatus. Examples of the portable-type ultrasound diagnosis apparatus may include, but are not limited to, a smartphone, a laptop computer, a personal digital assistant (PDA), and a tablet personal computer (PC), each of which may include a probe and an application.

The probe 20 may include a plurality of transducers. The plurality of transducers may transmit ultrasound signals to an object 10 in response to transmitting signals received from a transmitter 113. The plurality of transducers may receive ultrasound signals reflected from the object 10 to generate reception signals. In addition, the probe 20 and the ultrasound diagnosis apparatus 100 may be formed in one body, or the probe 20 and the ultrasound diagnosis apparatus 100 may be formed separately but linked wirelessly or via wires. In addition, the ultrasound diagnosis apparatus 100 may include one or more probes 20 according to embodiments.

The controller 120 controls the transmitter 113 for the transmitter 113 to generate transmitting signals to be applied to the plurality of transducers based on a position and a focal point of the plurality of transducers included in the probe 20.

The controller 120 controls an ultrasound receiver 115 to generate ultrasound data by converting reception signals received from the probe 20 from analogue to digital signals and summing the reception signals converted into digital form, based on a position and a focal point of the plurality of transducers.

The image processor 130 generates an ultrasound image by using the ultrasound data generated by the ultrasound receiver 115.

The display 140 may display the generated ultrasound image and various pieces of information processed by the ultrasound diagnosis apparatus 100. The ultrasound diagnosis apparatus 100 may include two or more displays 140 according to embodiments. Also, the display 140 may include a touchscreen in combination with a touch panel.

The controller 120 may control operations of the ultrasound diagnosis apparatus 100 and the flow of signals between internal elements of the ultrasound diagnosis apparatus 100. The controller 120 may include a memory for storing a program or data for performing functions of the ultrasound diagnosis apparatus 100 and a processor for processing the program or data. Also, the controller 120 may control an operation of the ultrasound diagnosis apparatus 100 by receiving a control signal from the input interface 170 or an external apparatus.

The ultrasound diagnosis apparatus 100 may include the communicator 160, and may be connected to external apparatuses (e.g., a server, a medical apparatus, and a portable device (e.g., a smartphone, a tablet personal computer (PC), or a wearable device)) via the communicator 160.

The communicator 160 may include at least one element capable of communicating with the external apparatuses. For example, the communicator 160 may include at least one among a short-range communication module, a wired communication module, and a wireless communication module.

The communicator 160 may transmit/receive a control signal and data to/from an external apparatus.

The storage 150 may store various data or programs for driving and controlling the ultrasound diagnosis apparatus 100, input/output ultrasound data, and the obtained ultrasound image.

The input interface 170 may receive a user's input for controlling the ultrasound diagnosis apparatus 100. Examples of the user's input may include, but are not limited to, inputs for manipulating buttons, keypads, mice, trackballs, jog switches, or knobs, inputs for touching a touchpad or a touchscreen, a voice input, a motion input, and a bio-information input (e.g., iris recognition or fingerprint recognition).

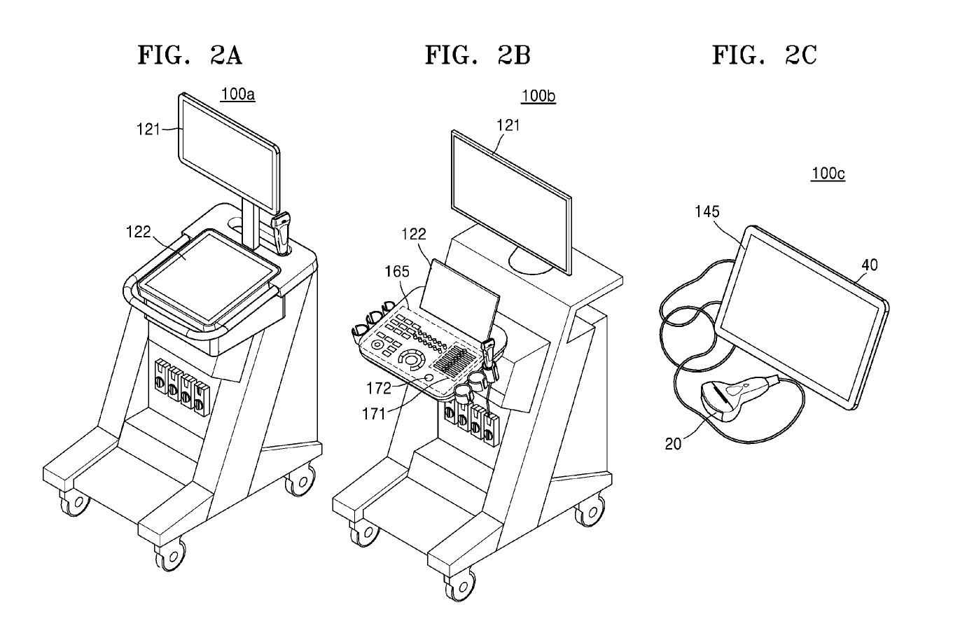

Examples of the ultrasound diagnosis apparatus 100 according to an embodiment will now be described with reference to FIGS. 2A, 2B, and 2C.

FIGS. 2A, 2B, and 2C are views illustrating ultrasound diagnosis apparatuses 100a, 100b, and 100c according to an embodiment.

Referring to FIGS. 2A and 2B, each of the ultrasound diagnosis apparatuses 100a and 100b may include a main display 121 and a sub-display 122. At least one of the main display 121 and the sub-display 122 may be a touchscreen. The main display 121 and the sub-display 122 may display ultrasound images or various information processed by the ultrasound diagnosis apparatuses 100a and 100b. The main display 121 and the sub-display 122 may be touchscreens, and may provide graphical user interfaces (GUIs), thereby receiving data for controlling the ultrasound diagnosis apparatuses 100a and 100b from a user. For example, the main display 121 may display an ultrasound image and the sub-display 122 may display a control panel for controlling display of the ultrasound image as a GUI. The sub-display 122 may receive control data for controlling display of an image through the control panel displayed as the GUI. The ultrasound diagnosis apparatuses 100a and 100b may control the display of the ultrasound image on the main display 121 by using the received control data.

Referring to FIG. 2B, the ultrasound diagnosis apparatus 100b may include a control panel 165 in addition to the main display 121 and the sub-display 122. The control panel 165 may include buttons, trackballs, jog switches, or knobs, and may receive data for controlling the ultrasound diagnosis apparatus 100 from the user. For example, the control panel 165 may include a time gain compensation (TGC) button 171 and a freeze button 172. The TGC button 171 is to set a TGC value for each depth of an ultrasound image. Also, when an input of the freeze button 172 is detected during scanning an ultrasound image, the ultrasound diagnosis apparatus 100b may keep displaying a frame image at that time point.

The buttons, trackballs, jog switches, and knobs included in the control panel 165 may be provided as a GUI on the main display 121 or the sub-display 122.

Referring to FIG. 2C, the ultrasound diagnosis apparatus 100c may be a portable-type ultrasound diagnosis apparatus. Examples of the portable-type ultrasound diagnosis apparatus may include, but are not limited to, a smartphone, a laptop computer, a PDA, and a tablet PC, each of which may include a probe and an application.

The ultrasound diagnosis apparatus 100c may include the probe 20 and a main body 40. The probe 20 may be connected to one side of the main body 40 by wire or wirelessly. The main body 40 may include a touchscreen 145. The touchscreen 145 may display an ultrasound image, various pieces of information processed by the ultrasound diagnosis apparatus 100c, and a GUI.

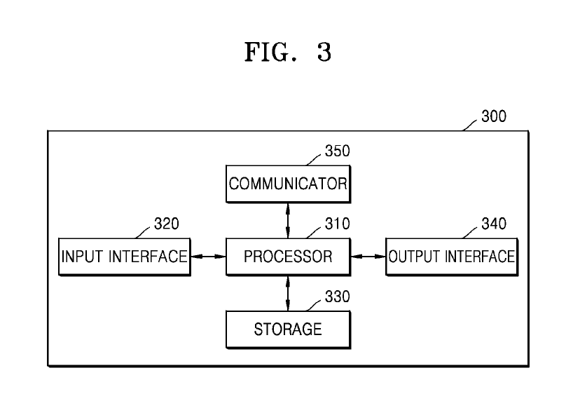

FIG. 3 is a block diagram illustrating a structure of an ultrasound imaging apparatus 300 according to an embodiment. The ultrasound imaging apparatus 300 may correspond to the ultrasound diagnosis apparatus 100 of FIG. 1. Also, the ultrasound imaging apparatus 300 may be implemented as any of the ultrasound diagnosis apparatuses 100a, 100b, and 100c of FIGS. 2A, 2B, and 2C.

As shown in FIG. 3, the ultrasound imaging apparatus 300 may include a processor 310, an input interface 320, a storage 330, an output interface 340, and a communicator 350. The processor 310 may correspond to the controller 120 of FIG. 1. Also, the processor 310 may include one or more processors. The input interface 320 may correspond to the input interface 170 of FIG. 1. The storage 330 may correspond to the storage 150 of FIG. 1. The output interface 340 may include the display 140 of FIG. 1. The communicator 350 may correspond to the communicator 160 of FIG. 1.

The storage 330 may store a patient list and information of patients in the patient list. According to an embodiment, the information of the patients may include at least one or a combination of information about diagnosis items of the patients, disability types of the patients, languages used by the patients, and caregivers of the patients. For example, the storage 330 may store information about patients who have auditory disability, use Korean, and undergo fetal ultrasound diagnosis.

The storage 330 may store a sign language, a subtitle, and a voice as auxiliary information corresponding to an ultrasound diagnosis process. The auxiliary information refers to a sign language, a subtitle, and a voice related to information corresponding to each progression stage of the ultrasound diagnosis process such as an action taken by a patient to undergo ultrasound diagnosis, an explanation about an image during an ultrasound scan, or an ultrasound diagnosis result in each progression stage of the ultrasound diagnosis process.

The input interface 320 may receive a user's input regarding patient information. For example, the input interface 320 may receive an input regarding new patient information (e.g., a diagnosis item, a disability type, and a used language) for adding a new patient to the patient list of the storage 330. Also, the input interface 320 may receive the user's input for additionally storing, in the storage 330, information about the patient of the patient list stored in the storage 330. For example, the input interface 320 may receive an input that adds information of the patient indicating that a language used by the patient is English to the patient information stored in the storage 330.

The input interface 320 may receive an input for changing an output form of the auxiliary information. For example, when the output form of the auxiliary information is a subtitle, the processor 310 may receive an input for changing the output method to a sign language.

The input interface 320 may receive the user's input regarding the auxiliary information corresponding to the ultrasound diagnosis process. According to an embodiment, the input interface 320 may receive an input that modifies the auxiliary information stored in the storage 330. According to another embodiment, the input interface 320 may receive an input that additionally stores, in the storage 330, auxiliary information corresponding to a new ultrasound diagnosis process. For example, when a varicose vein diagnosis process is added as a new ultrasound diagnosis process to the ultrasound imaging apparatus 300, the input interface 320 may receive an input that adds auxiliary information corresponding to the varicose vein diagnosis process.

The input interface 320 may receive an input including at least one or a combination of a voice and characters about a diagnosis situation and a diagnosis result corresponding to a progression stage of a diagnosis process during the ultrasound diagnosis process. For example, when the user performs fetal ultrasound diagnosis on a patient by using the ultrasound imaging apparatus 300, the input interface 320 may receive a voice or characters about a development state of a fetus as an input.

The processor 310 may determine an auxiliary information output form according to a disability type of a patient selected based on a patient selection input of the user. For example, when the selected patient is a person with auditory disability, the processor 310 may determine a sign language as the auxiliary information output form. Also, when the selected patient is a person with visual disability, the processor 310 may determine a voice as the auxiliary information output form.

When a sign language or a voice is included in the determined auxiliary output method, the processor 310 may determine that a language of an output subtitle or voice is a language used by the selected patient. For example, when the selected patient uses Korean, the processor 310 may determine that a language of a subtitle output when the auxiliary information output form is a subtitle is Korean.

The processor 310 may automatically execute the diagnosis process based on the patient information including a diagnosis item of the patient selected according to the user's patient selection input. For example, when the diagnosis item is thyroid ultrasound diagnosis according to the stored patient information of the selected patient, the processor 310 may automatically execute a process for the thyroid ultrasound diagnosis.

The processor 310 may change the output form of the auxiliary information based on an input for changing the output form of the auxiliary information. For example, when the output form of the auxiliary information is a subtitle, the processor 310 may change the output form of the auxiliary information from the subtitle to a sign language based on an input for changing the output method to the sign language.

The processor 310 may generate a sign language, a subtitle, and a voice as diagnosis information based on the input including at least one or a combination of a voice and characters about a diagnosis situation and a diagnosis result corresponding to the progression stage of the process during the ultrasound diagnosis process. For example, the processor 310 may generate a sign language, a subtitle, and a voice about a development state of a fetus as the diagnosis information based on the user's voice or character input regarding the development state of the fetus in the fetal ultrasound diagnosis process.

The output interface 340 may output the auxiliary information corresponding to the progression stage of the executed ultrasound diagnosis process, in the determined auxiliary information output form in real time. According to an embodiment, the output interface 340 may include a display and/or a speaker. For example, the thyroid ultrasound diagnosis process is being executed and a sign language is determined as the auxiliary information output form, the output interface 3540 may output a sign language corresponding to a progression stage of the thyroid ultrasound diagnosis process on the display in real time.

The communicator 350 may transmit/receive data between the ultrasound imaging apparatus 300 and an external apparatus.

According to an embodiment, the communicator 350 may transmit the auxiliary information corresponding to the ultrasound diagnosis process to a mobile terminal of a patient or a caregiver of the patient in real time. For example, the communicator 350 may transmit auxiliary information indicating that ‘Fetal heart rate is being executed’ in a fetal heart rate measuring step of the fetal ultrasound diagnosis process, in the output method determined by the processor 310, to the mobile terminal of the patient or the caregiver of the patient in real time.

According to an embodiment, the communicator 350 may transmit the diagnosis information generated by the processor 310 based on the input including at least one or a combination of a voice and characters about a diagnosis situation and a diagnosis result corresponding to the progression stage during the ultrasound diagnosis process a terminal (e.g., a smartphone, a wearable device, or a hearing aid) of the patient or the caregiver of the patient in real time. The diagnosis information may be transmitted in real time to the mobile terminal of the patient or the caregiver of the patient, in the output method determined by the processor 310 from among a sign language, a subtitle, and a voice.

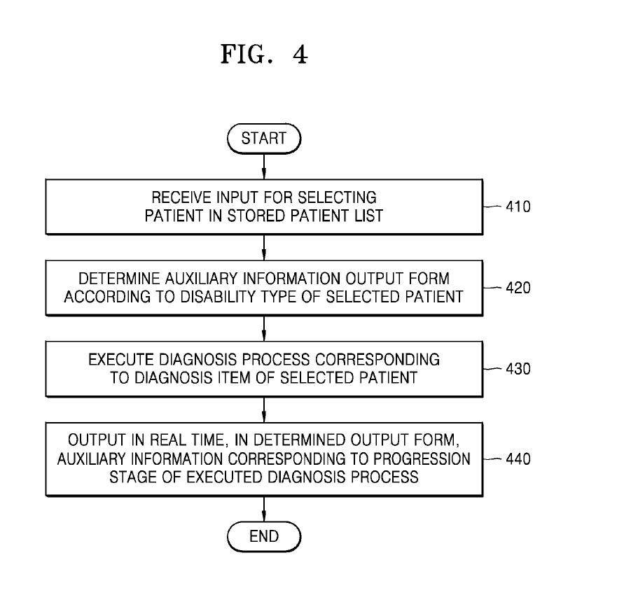

FIG. 4 is a flowchart of a method of automatically guiding ultrasound diagnosis performed by the ultrasound imaging apparatus 300, according to an embodiment.

A method of controlling an ultrasound image apparatus for ultrasound diagnosis may be performed by any of various types of ultrasound imaging apparatuses including a processor and an output interface and capable of processing an ultrasound image. Although the following will be described on the assumption that the method of controlling an ultrasound imaging apparatus is performed by one of the ultrasound diagnosis apparatuses 100, 100a, 100b, and 100c, and the ultrasound imaging apparatus 300, embodiments are not limited thereto. Also, the description made for the ultrasound diagnosis apparatuses 100, 100a, 100b, and 100c, and the ultrasound imaging apparatus 300 may apply to the method of controlling an ultrasound imaging apparatus.

In operation 410, the ultrasound imaging apparatus 300 receives an input that selects a patient in a patient list stored in the storage 330.

In operation 420, the ultrasound imaging apparatus 300 determines an auxiliary information output form according to a disability type of the selected patient based on a patient selection input.

In operation 430, the ultrasound imaging apparatus 300 executes an ultrasound diagnosis process corresponding to a diagnosis item of the selected patient.

In operation 440, the ultrasound imaging apparatus 300 outputs auxiliary information corresponding to a progression stage of the executed ultrasound diagnosis process, in the determined auxiliary information output form in real time.

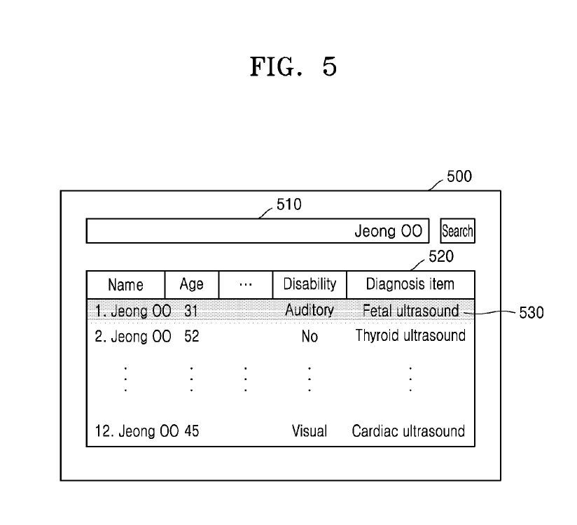

FIG. 5 is a diagram illustrating a process by which the ultrasound imaging apparatus 300 receives a patient's selection, according to an embodiment.

Referring to FIG. 5, the ultrasound imaging apparatus 300 may receive information about a patient according to a user input. According to an embodiment, the ultrasound imaging apparatus 300 displays a patient list stored in the storage 330 on a display of the output interface 340, and receives the patient's selection through the input interface 320. According to an embodiment, in order to provide a patient search function, the ultrasound imaging apparatus 300 provides a patient search box 510 through a graphical user interface (GUI) and receives a patient search input (e.g., Jeong 00) to be diagnosed by a user through the patient search box 510. The ultrasound imaging apparatus 300 outputs a patient list 520 corresponding to the patient search input. The ultrasound imaging apparatus 300 receives an input that selects a patient 530 in the output patient list 520 and determines the patient 530 selected by the user.

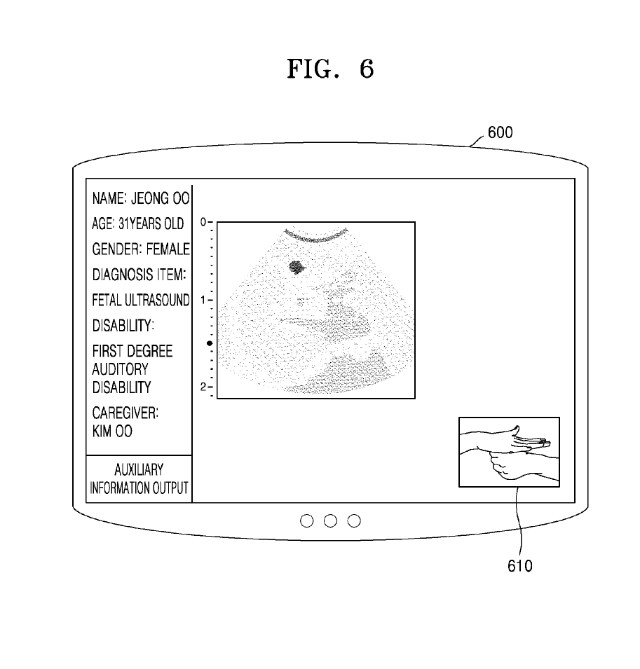

FIG. 6 is a diagram illustrating an example where when a selected patient is a person with auditory disability, the ultrasound imaging apparatus 300 automatically outputs auxiliary information, according to an embodiment.

FIG. 6 illustrates an example where when a diagnosis item of a selected patient is fetal ultrasound diagnosis and the selected patient is a person with auditory disability, auxiliary information is output. Referring to FIG. 6, the output interface 340 outputs patient information of the selected patent on a portion of a display 600. Since the diagnosis item of the selected patient is fetal ultrasound diagnosis, the processor 310 executes a fetal ultrasound diagnosis process. Also, since the selected patient is a person with auditory disability, the processor 310 determines a sign language as an auxiliary information output form.

Referring to FIG. 6, the output interface 340 outputs a sign language 610 corresponding to a progression stage of the fetal ultrasound diagnosis process on a portion of the display 600. The progression stage of the fetal ultrasound diagnosis process may be, for example, a fetal heart rate measuring step, and content of the sign language 610 that is auxiliary information may be ‘Fetal heart rate is being measured’.

When a selected patient is a person with auditory disability or the like, the ultrasound imaging apparatus 300 according to an embodiment automatically outputs auxiliary information corresponding to an executed ultrasound diagnosis process according to a disability type of the patient. Accordingly, according to an embodiment, even a disabled patient may be automatically guided about an ultrasound diagnosis process that is being performed, and thus may easily undergo ultrasound diagnosis.

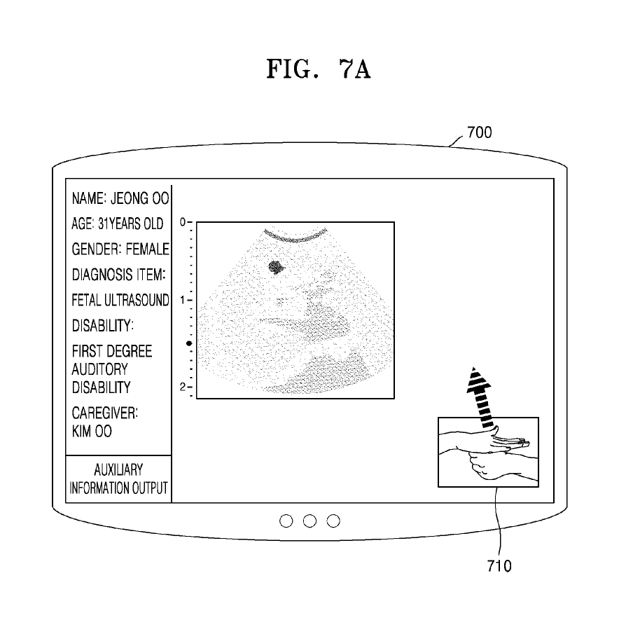

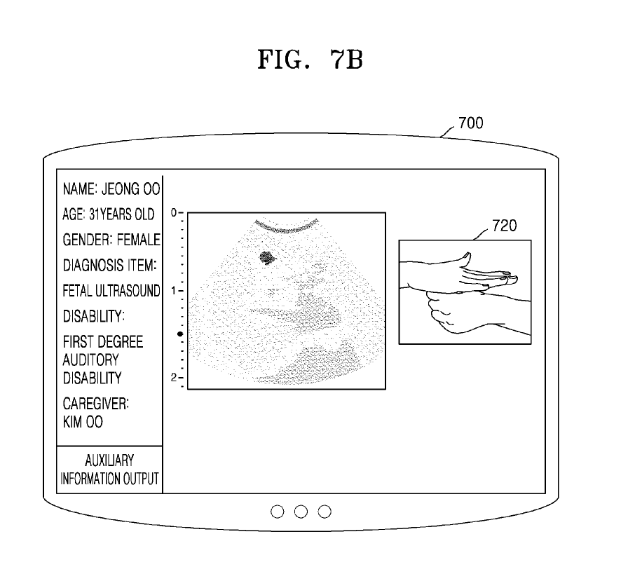

FIGS. 7A and 7B are diagrams illustrating an example where when output auxiliary information is a sign language or a subtitle, the ultrasound imaging apparatus 300 changes at least one of a position, a size, and a transparency of the output auxiliary information, according to an embodiment.

Referring to FIG. 7A, the ultrasound imaging apparatus 300 outputs an ultrasound image and a sign language 710 as auxiliary information on a display of a touchscreen 700. The ultrasound imaging apparatus 300 may receive an input regarding at least one or a combination of an output position, an output size, and an output transparency of the sign language 710 from a user.

According to an embodiment, the ultrasound imaging apparatus 300 may provide a user interface through the output interface 340 so that the user may change the output position, the output size, and the output transparency of the auxiliary information. For example, the ultrasound imaging apparatus 300 may provide an auxiliary information output size adjusting menu, an auxiliary information output position adjusting menu, and an auxiliary information output transparency adjusting menu. The user may adjust the output size of the auxiliary information by selecting an enlargement icon or a reduction icon of the auxiliary information output size adjusting menu.

According to an embodiment, the input interface 320 of the ultrasound imaging apparatus 300 may include the touchscreen 700, and may change a size and a position of the sign language 710 based on a touch input through the touchscreen 700. For example, the processor 310 may move a position of the sign language 710 to a second point and may display the sign language 710 at the second point on the touchscreen 700, based on a drag touch input that moves the sign language 710 from a first point to the second point. Also, the processor 310 may touch two points within or around an area where the sign language 710 is displayed, and may enlarge or reduce the sign language 710 and may display the enlarged or reduced sign language 710 on the touchscreen 700, based on a touch input that moves from the two touch points.

FIG. 7B is a diagram illustrating an example where a position of the sign language 710 of FIG. 7A is changed according to the user's drag input in an arrow direction and a sign language 720 obtained by enlarging the sign language 710 by using the user's two fingers is output.

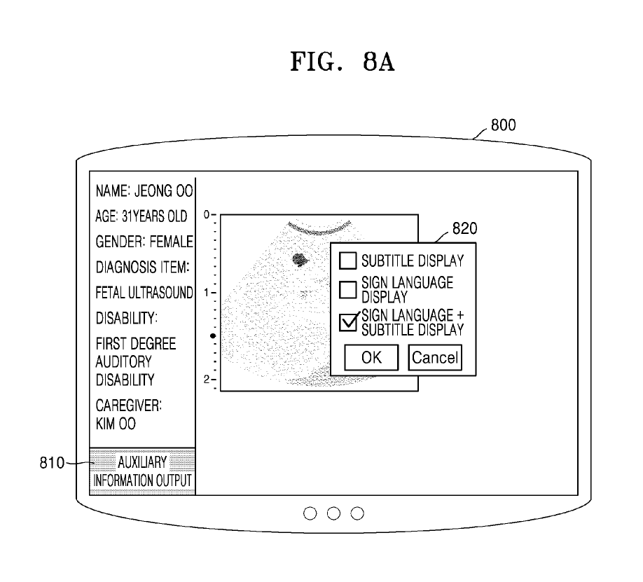

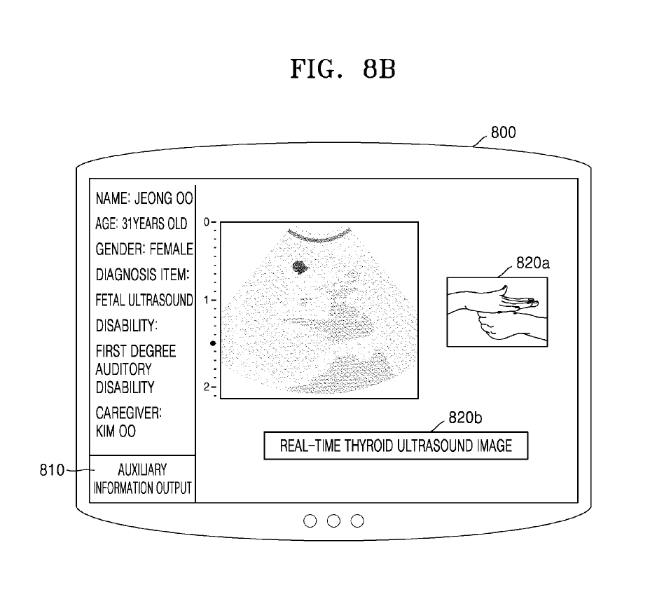

FIGS. 8A and 8B are diagrams illustrating an example where when a selected patient is a non-disabled person, the ultrasound imaging apparatus 300 outputs auxiliary information corresponding to an ultrasound diagnosis process, according to an embodiment.

FIG. 8A illustrates an example where the ultrasound imaging apparatus 300 receives a user's input regarding an auxiliary information output form according to an embodiment. According to an embodiment, when a selected patient is not a disabled person, the ultrasound imaging apparatus 300 may determine that the auxiliary information output form is to omit outputting auxiliary information. Even when the auxiliary information is not output, the ultrasound imaging apparatus 300 may provide a menu for selecting the auxiliary information output form.

For example, referring to FIG. 8A, the ultrasound imaging apparatus 300 outputs an auxiliary information output button 810 on a portion of a display 800. As the user's input regarding the auxiliary information output button 810 is received, the ultrasound imaging apparatus 300 outputs a menu 820 for selecting the auxiliary information output form on a portion of the display 800. The user may check a desired auxiliary information output form on the menu 820 for selecting the auxiliary information output form. For example, referring to FIG. 8A, an item ‘sign language+subtitle display’ is selected as the auxiliary information output form according to the user's check.

The ultrasound imaging apparatus 300 may output the auxiliary information based on the auxiliary information output form selected by the user. For example, referring to FIG. 8B, the ultrasound imaging apparatus 300 outputs a sign language 820a and a subtitle 820b corresponding to a thyroid ultrasound diagnosis process to portions of the display 800 based on the user's input that checks the item ‘sign language +subtitle display’ of FIG. 8A.

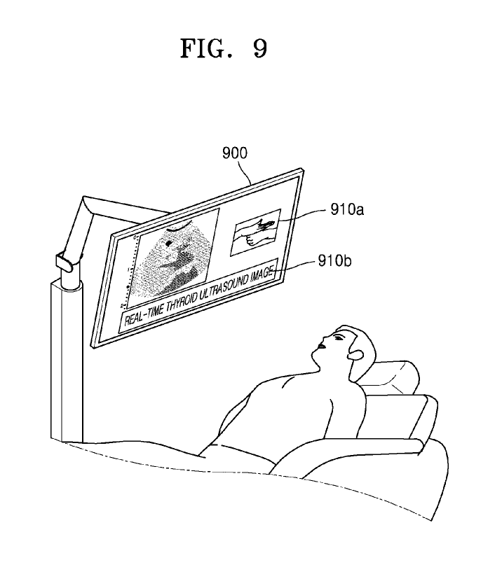

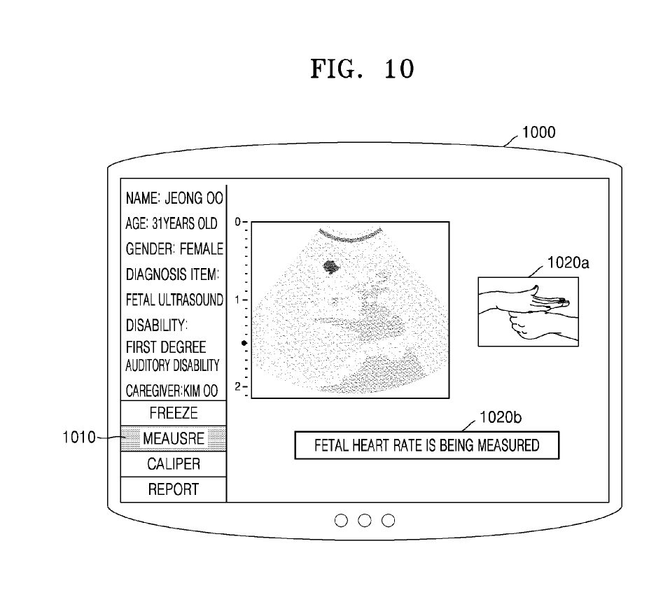

FIG. 9 is a diagram illustrating an example where the ultrasound imaging apparatus 300 including an auxiliary output interface 900 outputs auxiliary information (e.g., a sign language 910a or a subtitle 910b) corresponding to an ultrasound diagnosis process to the auxiliary output interface 900, according to an embodiment.

According to an embodiment, the auxiliary output interface 900 of the ultrasound imaging apparatus 300 may include a display. In this case, the auxiliary output interface 900 may be located in a place where a patient is able to see well within a field of view of the patient. The ultrasound imaging apparatus 300 may output, to the auxiliary output interface 900, the sign language 910a or the subtitle 910b as the auxiliary information output to the output interface 340.

It may be difficult for the patient to see the output interface 340 of the ultrasound imaging apparatus 300 according to a step of the ultrasound diagnosis process. Accordingly, according to an embodiment, the ultrasound imaging apparatus 300 enables the patient with auditory disability to more easily undergo ultrasound diagnosis by outputting the sign language 910a and the subtitle 910b as the auxiliary information through the auxiliary output interface 900 located within the field of view of the patient.

According to an embodiment, the auxiliary output interface 900 may include a speaker. The ultrasound imaging apparatus 300 may output the auxiliary information as a sound through the auxiliary output interface 900. When the auxiliary output interface 900 includes a speaker, the auxiliary output interface 900 may be located in a place where the patient is able to hear well within an audible range of the patient.

According to an embodiment, the ultrasound imaging apparatus 300 may transmit the auxiliary information corresponding to the ultrasound diagnosis process to a terminal (e.g., a smartphone, a wearable device, or a hearing aid) of the patient or a caregiver of the patient in real time. The auxiliary information transmitted in real time may be output in real time from the terminal of the patient or the caregiver of the patient. For example, the ultrasound imaging apparatus 300 may transmit the auxiliary information (e.g., the sign language 910a or the subtitle 910b) to a smartphone of the patient in real time, and the smartphone of the patient who receives the auxiliary information may display the auxiliary information on a screen. Alternatively, the ultrasound imaging apparatus 300 may transmit the auxiliary information to a hearing aid of the caregiver of the patient, and the hearing aid of the caregiver of the patient may output the received auxiliary information as a sound.

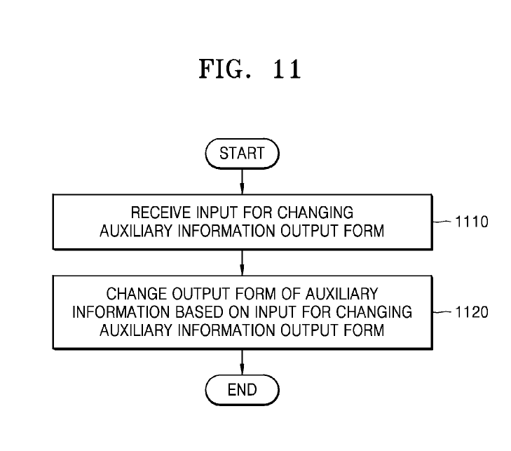

FIG. 10 is a diagram illustrating an example where whenever an event occurs in a progression stage of an ultrasound diagnosis process, the ultrasound imaging apparatus 300 outputs auxiliary information corresponding to the event, according to an embodiment.

The ultrasound imaging apparatus 300 may receive a user's input regarding an ultrasound diagnosis event in an ultrasound diagnosis process. The ultrasound diagnosis event may include at least one or a combination of a freeze, a measurement, a caliper, and a report.

Referring to FIG. 10, the ultrasound imaging apparatus 300 executes a fetal ultrasound diagnosis process. The ultrasound imaging apparatus 300 receives a measurement event input when the user clicks on a measurement event button 1010. The ultrasound imaging apparatus 300 executes a measurement event based on the measurement event input, and outputs a sign language 1020a and a subtitle 1020b corresponding to the measurement event to portions of a display 1000.

According to an embodiment, even when a patient is a disabled person, since the patient receives auxiliary information about an event occurring in a progression stage of an ultrasound diagnosis process in real time, the patient may easily undergo ultrasound diagnosis.

FIG. 11 is a flowchart of a method by which the ultrasound imaging apparatus 300 changes an output method of auxiliary information, according to an embodiment.

In operation 1110, the ultrasound imaging apparatus 300 receives an input for changing an auxiliary information output form. For example, when an output form of output auxiliary information is a subtitle, the ultrasound imaging apparatus 300 may receive an input for changing the output method to a sign language.

In operation 1120, the ultrasound imaging apparatus 300 changes the output form of the auxiliary information based on the input for changing the auxiliary information output form. For example, the ultrasound imaging apparatus 300 may change the output form of the auxiliary information from a subtitle to the sign language.

Embodiments may be implemented on a computer-readable recording medium storing instructions and data executable by computers. The instructions may be stored as program codes, and when being executed by a processor, may cause a predetermined program module to be generated and a predetermined operation to be performed. Also, when executed by the processor, the instructions may cause predetermined operations of the embodiments to be performed.

While one or more embodiments have been described with reference to the figures, it will be understood by one of ordinary skill in the art that various changes in form and details may be made therein without departing from the spirit and scope of the disclosure as defined by the following claims.

What is claimed is:

1. An ultrasound imaging apparatus comprising:
a storage configured to store a patient list and store a sign language, a subtitle, and a voice as auxiliary information corresponding to a diagnosis process;
an input interface configured to receive an input for selecting a patient in the patient list;
at least one processor configured to determine an auxiliary information output form according to a disability type of the selected patient, and execute a diagnosis process corresponding to a diagnosis item of the selected patient; and
an output interface configured to output in real time, in the determined auxiliary information output form, auxiliary information corresponding to a progression stage of the executed diagnosis process,
wherein, when the selected patient is a person with auditory disability, the sign language is determined as the auxiliary information output form.

2. The ultrasound imaging apparatus of claim 1, wherein the input interface is further configured to receive an input regarding patient information, and the storage is further configured to store the patient information in the patient list based on the input regarding the patient information,
wherein the patient information comprises at least one or a combination of information about a disability type of the patient, information about a language used by the patient, and information about a caregiver of the patient.

3. The ultrasound imaging apparatus of claim 1, wherein the input interface is further configured to receive an input for changing an output form of the auxiliary information, and
the at least one processor is further configured to change the output form of the auxiliary information based on the input for changing the output form of the auxiliary information,
wherein the input for changing the output form of the auxiliary information comprises an input for stopping outputting of the auxiliary information.

4. The ultrasound imaging apparatus of claim 1, wherein, when the determined auxiliary information output form is the sign language, the subtitle, or a combination thereof,
the input interface is further configured to receive an input regarding at least one or a combination of an output position, an output size, and an output transparency of the auxiliary information corresponding to the progression stage of the executed diagnosis process, and
the output interface is further configured to output the auxiliary information corresponding to the progression stage of the executed diagnosis process based on the input regarding the at least one or the combination of the output position, the output size, and the output transparency of the auxiliary information.

5. The ultrasound imaging apparatus of claim 1, wherein, when the selected patient is a non-disabled person,
the input interface is further configured to receive a user input regarding the auxiliary information output form,
the at least one processor is further configured to change the determined auxiliary information output form based on the user input regarding the auxiliary information output form, and
the output interface is further configured to output in real time, in the changed auxiliary information output form, the auxiliary information corresponding to the progression stage of the executed diagnosis process.

6. The ultrasound imaging apparatus of claim 1, further comprising an auxiliary output interface configured to output in real time, in the determined auxiliary information output form, the auxiliary information corresponding to the progression stage of the executed diagnosis process.

7. The ultrasound imaging apparatus of claim 1, further comprising a communicator configured to transmit in real time, in the determined auxiliary information output form, the auxiliary information corresponding to the progression stage of the executed diagnosis process to a mobile terminal of the selected patient or a mobile terminal of a caregiver of the selected patient.

8. The ultrasound imaging apparatus of claim 1, wherein the output interface is further configured to, whenever an event occurs in the progression stage of the executed diagnosis process, output in real time, in the determined auxiliary information output form, auxiliary information corresponding to the event,
wherein the event comprises at least one or a combination of a freeze, a measurement, a caliper, and a report.

9. The ultrasound imaging apparatus of claim 1, wherein the input interface is further configured to receive a user input regarding the auxiliary information corresponding to the diagnosis process, and
the storage is further configured to store the sign language, the subtitle, and the voice as the auxiliary information corresponding to the diagnosis process, based on the user input regarding the auxiliary information corresponding to the diagnosis process,
wherein the user input regarding the auxiliary information corresponding to the diagnosis process comprises at least one or a combination of an input for modifying the auxiliary information stored in the storage and an input for adding the auxiliary information corresponding to the diagnosis process.

10. The ultrasound imaging apparatus of claim 1, wherein the input interface is further configured to receive an input comprising at least one or a combination of a voice and characters regarding a diagnosis situation and a diagnosis result, the diagnosis situation and the diagnosis result each corresponding to the progression stage of the diagnosis process,
the at least one processor is further configured to generate the sign language, the subtitle, or the voice as diagnosis information, based on the input, and the output interface is further configured to output the diagnosis information in the determined auxiliary information output form in real time.

11. The ultrasound imaging apparatus of claim 10, further comprising a communicator configured to transmit the generated diagnosis information in the determined auxiliary information output form to a mobile terminal of the selected patient or a mobile terminal of a caregiver of the selected patient.

12. A method of controlling an ultrasound imaging apparatus, the method comprising:
receiving an input for selecting a patient in a stored patient list;
determining an auxiliary information output form according to a disability type of the selected patient;
executing a diagnosis process corresponding to a diagnosis item of the selected patient; and
outputting in real time, in the determined auxiliary information output form, auxiliary information corresponding to a progression stage of the executed diagnosis process,
wherein, when the selected patient is a person with auditory disability, a sign language is determined as the auxiliary information output form.

13. The method of claim 12, further comprising:
receiving an input regarding patient information; and
storing the patient information in the patient list based on the input regarding the patient information,
wherein the patient information comprises at least one or a combination of information about a disability type of the patient, a language used by the patient, and a caregiver of the patient.

14. The method of claim 12, further comprising:
receiving an input for changing an output form of the auxiliary information; and
changing the output form of the auxiliary information based on the input for changing the output form of the auxiliary information,
wherein the input for changing the output form of the auxiliary information comprises an input for stopping outputting of the auxiliary information.

15. The method of claim 12, wherein, when the determined auxiliary information output form is the sign language, a subtitle, or a combination thereof, the method further comprises:
receiving an input regarding at least one or a combination of an output position, an output size, and an output transparency of the auxiliary information corresponding to the progression stage of the executed diagnosis process; and
outputting the auxiliary information corresponding to the progression stage of the executed diagnosis process, based on the input regarding the at least one or the combination of the output position, the output size, and the output transparency of the auxiliary information.

16. The method of claim 12, wherein, when the selected patient is a non-disabled person, the method further comprises:
receiving a user input regarding the auxiliary information output form; and
changing the determined auxiliary information output form based on the user input regarding the auxiliary information output form,
wherein the outputting of the auxiliary information corresponding to the progression stage of the executed diagnosis process comprises outputting the auxiliary information in the changed auxiliary information output form in real time.

17. The method of claim 12, further comprising transmitting in real time, in the determined auxiliary information output form, the auxiliary information corresponding to the progression stage of the executed diagnosis process to a mobile terminal of the selected patient or a mobile terminal of a caregiver of the selected patient.

18. The method of claim 12, wherein the outputting of the auxiliary information comprises, whenever an event occurs in the progression stage of the executed diagnosis process, outputting in real time, in the determined auxiliary information output form, auxiliary information corresponding to the event,
wherein the event comprises at least one or a combination of a freeze, a measurement, a caliper, and a report.

19. The method of claim 12, further comprising:
receiving a user input regarding the auxiliary information corresponding to the diagnosis process; and
storing the sign language, a subtitle, and a voice as the auxiliary information corresponding to the diagnosis process, based on the user input regarding the auxiliary information corresponding to the diagnosis process,
wherein the user input regarding the auxiliary information corresponding to the diagnosis process comprises at least one or a combination of an input for modifying stored auxiliary information and an input for adding the auxiliary information corresponding to the diagnosis process.

20. A computer program product comprising a computer-readable storage medium comprising instructions for performing the method of claim 12.
